(19) United States
(12) Patent Application Publication
(10) Pub. No.: US 2016/0103938 A1
(43) Pub. Date: Apr. 14, 2016

(54) DIAMOND PROCESSING

(71) Applicant: Trifecta Industries Ltd., Vancouver (CA)

(72) Inventor: Aaron Ariel, Vancouver BC (CA)

(21) Appl. No.: 14/732,152

(22) Filed: Jun. 5, 2015

Related U.S. Application Data
(60) Provisional application No. 62/062,785, filed on Oct. 10, 2014.

(51) Int. Cl. G06F 17/50; G06Q 30/02; G01N 33/38

(52) U.S. Cl. CPC G06F 17/5009; G01N 33/381; G06Q 30/0206

(57) ABSTRACT

Technology is disclosed for use in diamond processing. Diamond parameter information is obtained and information for numerous diamonds can be aggregated into a database. At least one candidate diamond for potential recutting is identified based on processing the received measurement information by applying logic based on selection criteria stored in the memory. A virtual model is then generated of the at least one candidate diamond based, at least in part, on the received measurement information. The virtual model is analyzed by applying recut criteria to determine a parameter indicative of recut potential and this is output for use in selecting diamonds for a recutting operation. By reducing the requirement to transport diamonds to an analysis site, significant energy, processing, and environmental benefits may be achieved.

### CROSS-REFERENCE TO RELATED APPLICATION

This application hereby claims priority to U.S. Provisional Patent Application No. 62/062,785, filed Oct. 10, 2014, which is incorporated herein by reference in its entirety.

### TECHNICAL FIELD

The present technology generally relates to material processing, more specifically to processing of diamonds.

### BACKGROUND

Rough diamonds obtained directly from diamond mining operations are manufactured into polished diamonds for resale by cutting operations. Initial cutting may not always yield optimum results and, in certain instances, it can be advantageous to recut already polished diamonds. This can be the case if the original manufacturer has made imperfect decisions while manufacturing the diamond

Quite unlike most other material handling and processing problems, diamond cutting and processing is a technically difficult process. Diamond cutting and processing involves highly specialized tools operating on rare and expensive raw materials with individually varying attributes with minimal practical scope for trial and error during the material processing operation. Thus, analyzing diamonds to identify appropriate cutting to be done and appropriate candidates for cutting is an important part of the overall material processing operation. One difficulty in this process is identifying promising candidate diamonds for recutting. Traditionally this has been more of an art than a science involving human experts. There has, however, been some automation. For example, machines such as the DiaExpert™ offered by Sarine Technologies Ltd. provide automated measurement and analysis. After physically measuring diamonds and analyzing them, a significant number will likely prove to be poor candidates for recutting. Conventional human and automated approaches are both wasteful—a significant proportion of raw materials are rejected, with associated transport and storage space and energy consumption as well as acquisition and resale costs. These handling problems are greatly exacerbated by the practical issues of dealing securely with highly valuable raw materials (diamonds), which means that in the real world transporting an item of raw material weighing only a gram or so consumes a disproportionately large amount of energy.

### BRIEF DESCRIPTION OF THE DRAWINGS

FIG. 1 is a block diagram illustrating an overview of devices on which some embodiments of the disclosed technology can operate.

FIG. 2 is a block diagram illustrating an overview of an environment in which some embodiments of the disclosed technology can operate.

FIG. 3 is a block diagram illustrating components which, in some implementations, can be used in a system employing the disclosed technology.

FIG. 4 is a flow diagram illustrating a process for evaluating diamonds for recut potential in accordance with some embodiments of the present technology.

FIG. 5 illustrates an example diamond certification document.

FIG. 6 illustrates a graphical user interface of a system for generating a virtual model of a diamond in accordance with an embodiment of the present technology.

FIG. 7 illustrates another graphical user interface of a system for evaluating recut potential of a diamond in accordance with an embodiment of the present technology.

### DETAILED DESCRIPTION

The present technology is directed to systems and methods for processing diamonds. In particular, embodiments of the present technology described herein can analyze diamonds for recut potential without physically measuring and analyzing each diamond. This allows for a more effective selection of diamonds for recutting. For example, a virtual model can be constructed for a diamond. This virtual model can then be analyzed for recut potential, allowing a more informed decision in selecting candidate diamonds for recutting.

Thus, unlike prior art human or automated approaches, recut analysis according to the present technology is performed not directly on the diamond itself but on a virtual model of the diamond. This stems from a realization pursuant to the technology that, rather than trying to provide tools to assist or mimic the traditional human evaluation approach, a different technical approach that does not directly use the diamond at the point of analysis may improve the overall processing operation. The parameters can be obtained over a network and the recut evaluation can also be output over a network. Thus, a number of diamonds can be rapidly evaluated from remote sources without the issues of transporting the raw materials to an evaluation point, and only those selected for a cutting operation need to be acquired and transported to the cutting operation site. Although logic rules may perform analysis automatically, embodiments may allow for or include operator input in the evaluation and selection modules or may include further final evaluation based on the stored model.

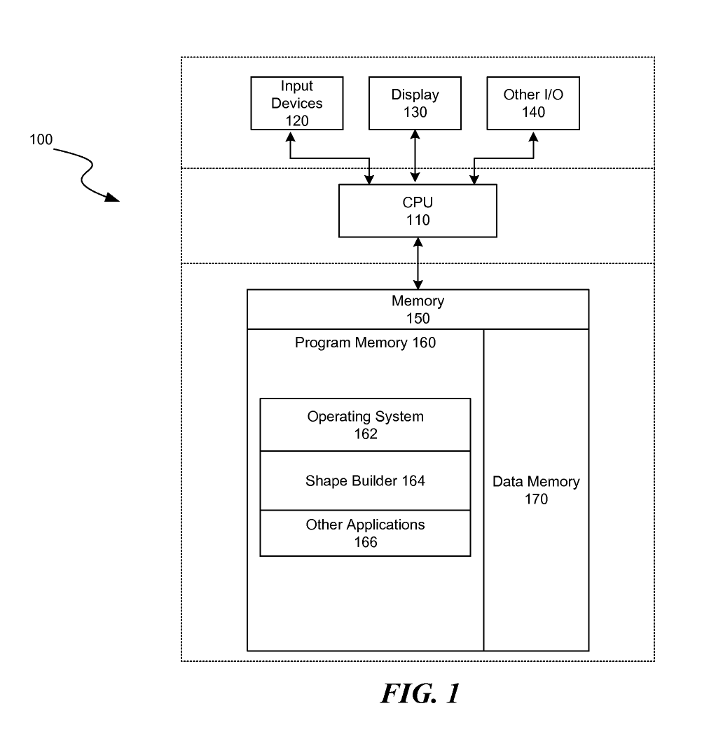

Several embodiments of the described technology are discussed below in more detail in reference to the figures. Turning now to the figures, for example, FIG. 1 is a block diagram illustrating an overview of devices 100 on which some embodiments of the disclosed technology may operate. The devices may comprise hardware components of a device 100 for operating diamond evaluation system. The device 100, for example, includes one or more input devices 120 that provide input to the CPU (processor) 110, notifying it of actions performed by a user. The actions are typically mediated by a hardware controller that interprets the signals received from the input device and communicates the information to the CPU 110 using a communication protocol. Input devices 120 include, for example, a mouse, keyboard, a touchscreen, an infrared sensor, a touchpad, a wearable input device, a camera- or image-based input device, a microphone, or other user input devices.

The CPU 110 can be a single processing unit or multiple processing units in a device or distributed across multiple devices. CPU 110 can be coupled to other hardware devices, for example, with the use of a BUS, such as a PCI BUS or SCSI BUS. The CPU 110 can communicate with a hardware controller for devices, such as for a display 130. Display 130 can be used to display text and graphics. In some examples, display 130 provides graphical and textual visual feedback to a user. In some implementations, the display includes the input device as part of the display, such as when the input device is a touchscreen or is equipped with an eye direction monitoring system. In some implementations, the display is separate from the input device. Examples of display devices include an LCD display screen, an LED display screen, a projected display (such as a heads-up display device or a head-mounted device), and so on. Other I/O devices 140 can also be coupled to the processor, such as a network card, video card, audio card, USB, firewire or other external devices, camera, printer, speakers, CD-ROM drive, DVD drive, disk drives, or Blu-Ray devices.

In some implementations, the device 100 also includes a communication device capable of communicating wirelessly or wire-based with a network node. The communication device can communicate with another device or a server through a network using, for example, TCP/IP protocols. For example, device 100 can utilize the communication device to distribute operations across multiple network devices.

The CPU 110 has access to a memory 150. A memory includes one or more of various hardware devices for volatile and non-volatile storage, and can include both read-only and writable memory. For example, a memory can comprise random access memory (RAM), CPU registers, read-only memory (ROM), and writable non-volatile memory, such as flash memory, hard drives, floppy disks, CDs, DVDs, magnetic storage devices, tape drives, device buffers, and so forth. A memory is not a propagating signal divorced from underlying hardware; a memory is thus non-transitory. Memory 150 includes program memory 160 that stores programs and software, such as an operating system 162, shape builder 164, and any other application programs 166.

The disclosed technology is operational with numerous other general purpose or special purpose computing system environments or configurations. Examples of well-known computing systems, environments, and/or configurations that may be suitable for use with the technology include, but are not limited to, personal computers, server computers, handheld or laptop devices, cellular telephones, wearable electronics, tablet devices, multiprocessor systems, microprocessor-based systems, set-top boxes, programmable consumer electronics, network PCs, minicomputers, mainframe computers, distributed computing environments that include any of the above systems or devices, and the like.

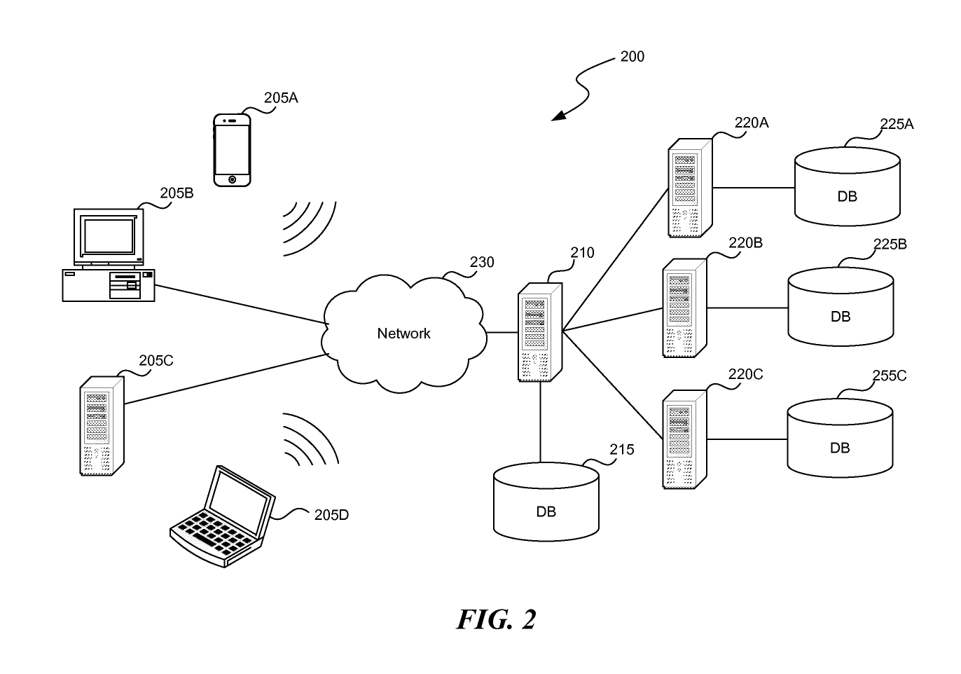

FIG. 2 is a block diagram 200 illustrating an overview of an environment in which some embodiments of the disclosed technology may operate. An environment for implementing the technology can include one or more client computing devices 205A-D, examples of which may include device 100. Client computing devices 205 can operate in a networked environment using logical connections through network 230 to one or more remote computers such as server computing device.

In some implementations, server 210 can be an edge server which receives client requests and coordinates fulfillment of those requests through other servers, such as servers 220A-C. Server computing devices 210 and 220 can comprise computing systems, such as device 100. Though each server computing device 210 and 220 is displayed logically as a single server, server computing devices can each be a distributed computing environment encompassing multiple servers located at the same or at geographically disparate physical locations. In some implementations, each server 220 corresponds to a group of servers.

Client computing devices 205 (“clients”) and server computing devices 210 and 220 (“servers”) can each act as a server or client to other server/client devices. Server 210 can connect to a database 215. Database 215 can warehouse information such as wholesale diamond lists, measurements or other characteristics of diamonds, information relating to virtual models of diamonds, diamond market information (e.g., fair market value for diamonds depending on various features), etc.

Servers 220A-C can each connect to a corresponding database 225A-C. As discussed above, each server 220 may correspond to a group of servers, and each of these servers can share a database or can have their own database. Though databases 215 and 225 are displayed logically as single units, databases 215 and 225 can each be a distributed computing environment encompassing multiple servers, can be located within their corresponding server, or can be located at the same or at geographically disparate physical locations.

Network 230 can be a local area network (LAN) or a wide area network (WAN), but can also be other wired or wireless networks. Network 230 may be the internet or some other public or private network. The client computing devices 205 can be connected to network 230 through a network interface, such as by wired or wireless communication. While the connections between server 210 and servers 220 are shown as separate connections, these connections can be any kind of local, wide area, wired, or wireless network, including network 230 or a separate public or private network.

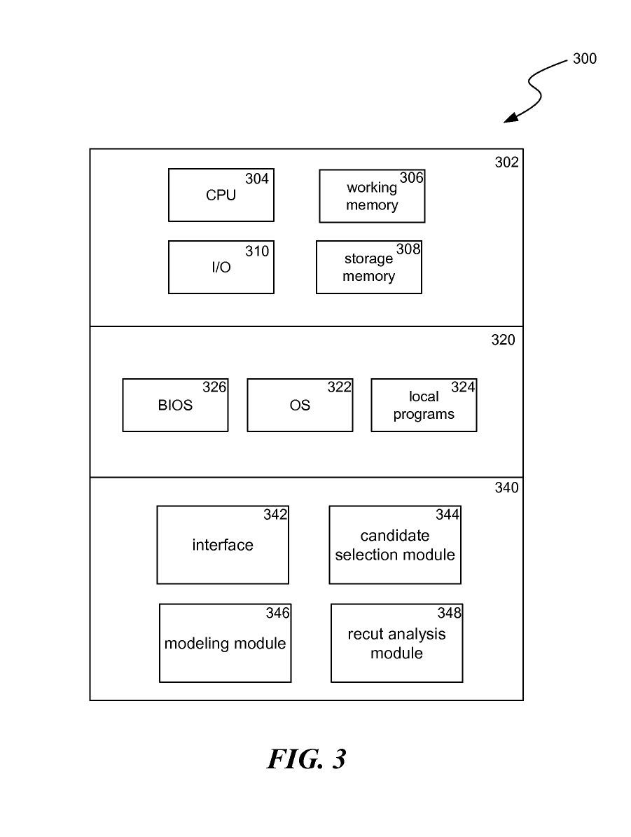

FIG. 3 is a block diagram illustrating components 300 which, in some implementations, can be used in a system implementing the disclosed technology. The components 300 include hardware 302, general software 320, and specialized components 340. As discussed above, a system implementing the disclosed technology can use various hardware including central processing units 304, working memory 306, storage memory 308, and input and output devices 310. Components 300 can be implemented in a client computing device such as client computing devices 205 or on a server computing device, such as server computing device 210 or 220.

General software 320 can include various applications including an operating system 322, local programs 324, and a BIOS 326. Specialized components 340 can be subcomponents of a general software application 320, such as a local program 324. Specialized components 340 can include a candidate selection module 344, a modeling module 346, and a recut analysis module 348, and components which can be used for controlling and receiving data from the specialized components, such as interface 342. In some implementations, components 300 can be distributed across multiple computing systems or can include an interface to a server-based application.

The candidate selection module 344 can retrieve diamond inventory information from online wholesale diamond websites. The candidate selection module 344 can search databases of diamond inventory information to filter for diamonds that match a set of parameters that make them likely to be good candidates for recut. Once at least one recut candidate diamond is identified, the measurements corresponding to the identified candidate diamond are obtained. In certain cases, some information about the identified candidate diamond may be missing. In some embodiments, the candidate selection module 344 can calculate or extrapolate the missing measurements based upon the measurements which have been provided. Once the candidate selection module 344 has identified at least one candidate diamond and has obtained a full listing of required measurements, this information can be transmitted to the modeling module 346.

The modeling module 346 can use the measurements of the identified candidate diamond to generate a 3D model. The virtual model of the diamond can be a projected approximation of the real diamond, similar to what would be obtained by physical measurement of the diamond using the Sarine DiaExpert™ or other such device.

The recut analysis module 348 can then evaluate the model generated by the modeling module 346. For example, particular instructions regarding cut and symmetry parameters can be provided. The recut analysis module 348 can then evaluate virtual model 601 to project the weight of the recut polished diamond given those particular constraints. In some embodiments, the proposed recut can be calculated automatically based upon a variety of selected parameters, such as symmetry, desired cut, etc. In addition to technical analysis the recut analysis module 348 may also use other stored criteria to estimate a fair market value of the candidate diamond after the proposed recut, for example based on estimated recut weight and other shape factors.

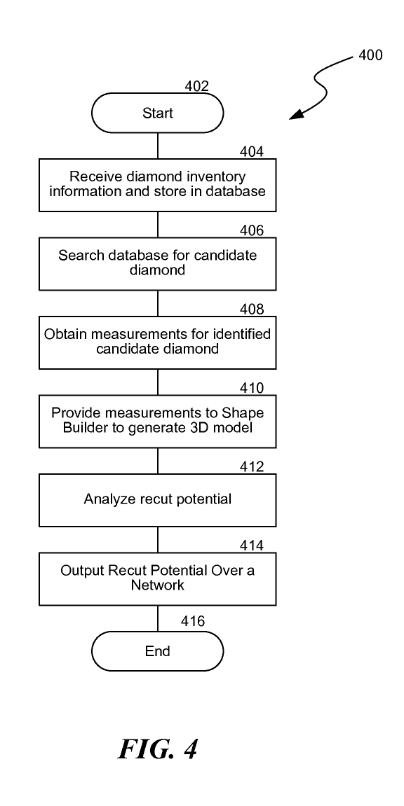

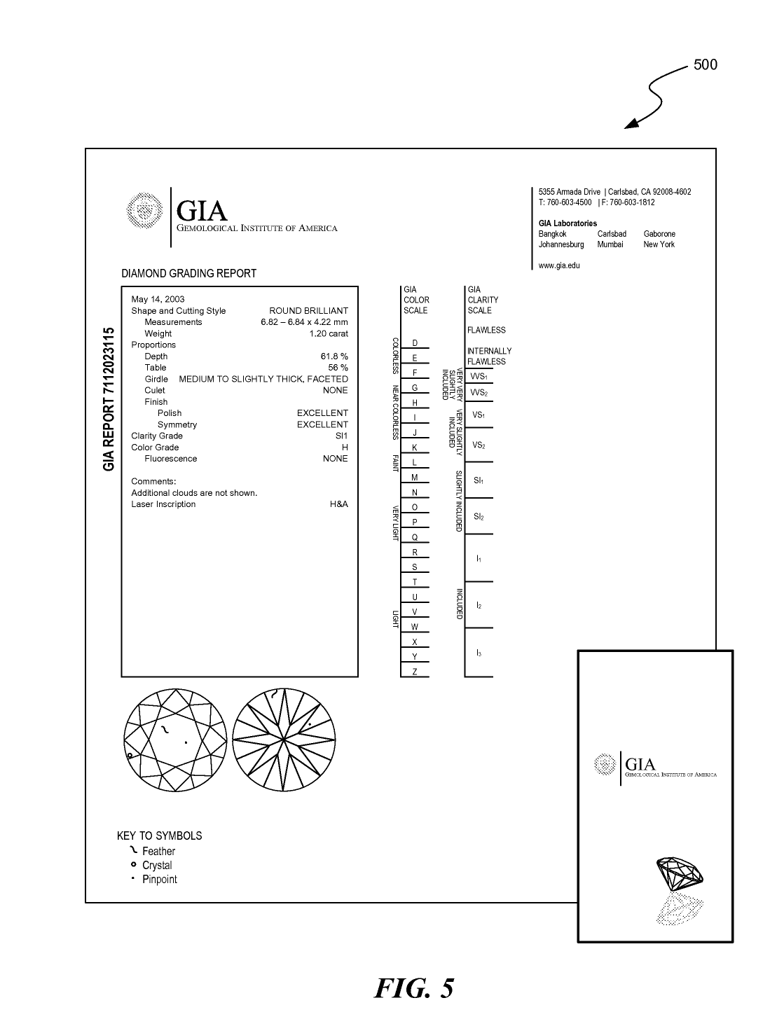

FIG. 4 is a flow diagram illustrating a process used in some embodiments for evaluating diamonds for recut potential. Routine 400 begins in block 402. In block 404, diamond inventory information is received and stored in a database. For example, diamond information can be provided by a third-party certifier, such as the Gemological Institute of America (GIA) or other entity. FIG. 5, for example, illustrates an example GIA diamond certificate 500, which includes information about a particular diamond such as dimensions, angles and qualitative assessments of the diamond's cut grade, polish, and symmetry. For example, in some embodiments the various qualitative assessments can identify the diamond as poor (P), Fair (F), Good (G), Very Good (VG), or Excellent (EX). The qualifications assigned by the certifier are an important consideration when buying or selling diamonds as they can determine the diamond's fair market value. A diamond with Excellent grades in cut, polish, and symmetry (known as a “Triple Excellent”) is often worth more than a bigger diamond of poorer cut, polish, and symmetry grades. Thus, when evaluating a diamond at the end of the manufacturing process, a manufacturer may decide to recut a diamond and sacrifice some carat weight in the process to achieve a better overall grade in cut, polish, or symmetry. Such considerations may be combined with other technical criteria for evaluating a recut.

The measurements of the diamond depicted on the certificate as shown in FIG. 5 generally reflect a broad representation rather than specific measurements which might be available after individually scanning the diamond using a diamond evaluating device such as the Sarine DiaExpert™ or other such device. The certifier may depict average measurement values, whereas more detailed physical analysis may provide individual measurements. As one example, the angle between the girdle (widest part of the diamond) and the facets directly above the girdle (“crown facets”) is called the “crown angle.” In reality, the crown angle is often not perfectly uniform around the diamond. The certifier may not reflect this variation in its reporting of the diamond dimensions, but more detailed physical analysis can capture these variances around the diamond.

The information obtained from the certifier or other entity can be received electronically over a network connection, for example. In some embodiments, for example, a first script can retrieve diamond inventory information from online wholesale diamond websites. In some embodiments, any inventory listing of diamonds provided by diamond suppliers or any subscription daily feed of inventory sent directly by diamond suppliers may also be used as a source of diamond inventory information. The first script can aggregate the various diamond inventory information into a database for further analysis.

Routine 400 continues in block 404 by searching the database for a candidate diamond. For example, a second script can search the database to filter for diamonds that match a set of parameters that make them likely to be profitably recut. Once at least one recut candidate diamond is identified, the measurements corresponding to the identified candidate diamond are obtained from the database in block 406. In certain cases, some information about the identified candidate diamond may be missing. In some embodiments, a third script can calculate or extrapolate the missing measurements based upon the measurements which have been provided. In some embodiments, the third script can pull measurements from an associated laboratory certificate (e.g., the GIA certificate illustrated in FIG. 5). In some embodiments, missing information can be obtained from another source, for example a laboratory web-based API through which the diamond can be identified by searching.

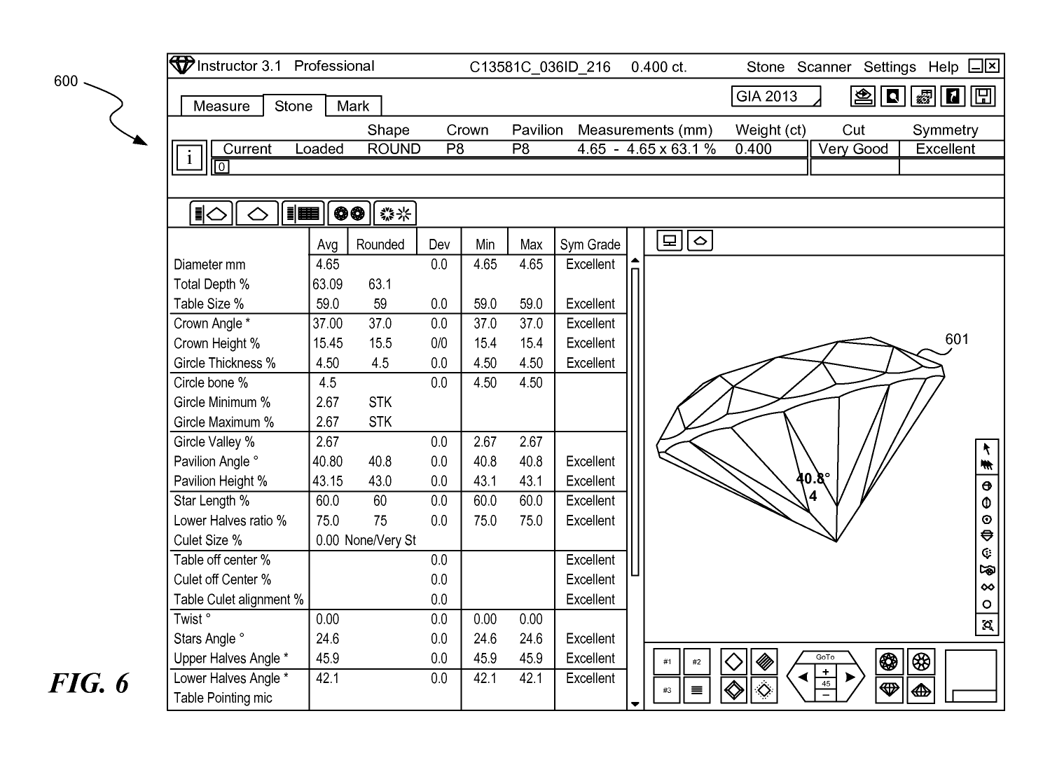

At this stage, at least one candidate diamond has been identified and a full listing of required measurements have been obtained. In block 410, the measurements of the identified candidate diamond are provided to the shape builder system which generates a 3D model. FIG. 6, for example, illustrates a graphical user interface 600 for the shape builder system. As illustrated, a 3D virtual model 601 of the identified candidate diamond can be generated based on the obtained measurements and displayed to the user. The virtual model 601 is a projected approximation of the real diamond, similar to what would be obtained by physical measurement of the diamond using the Sarine DiaExpert™ or other such device.

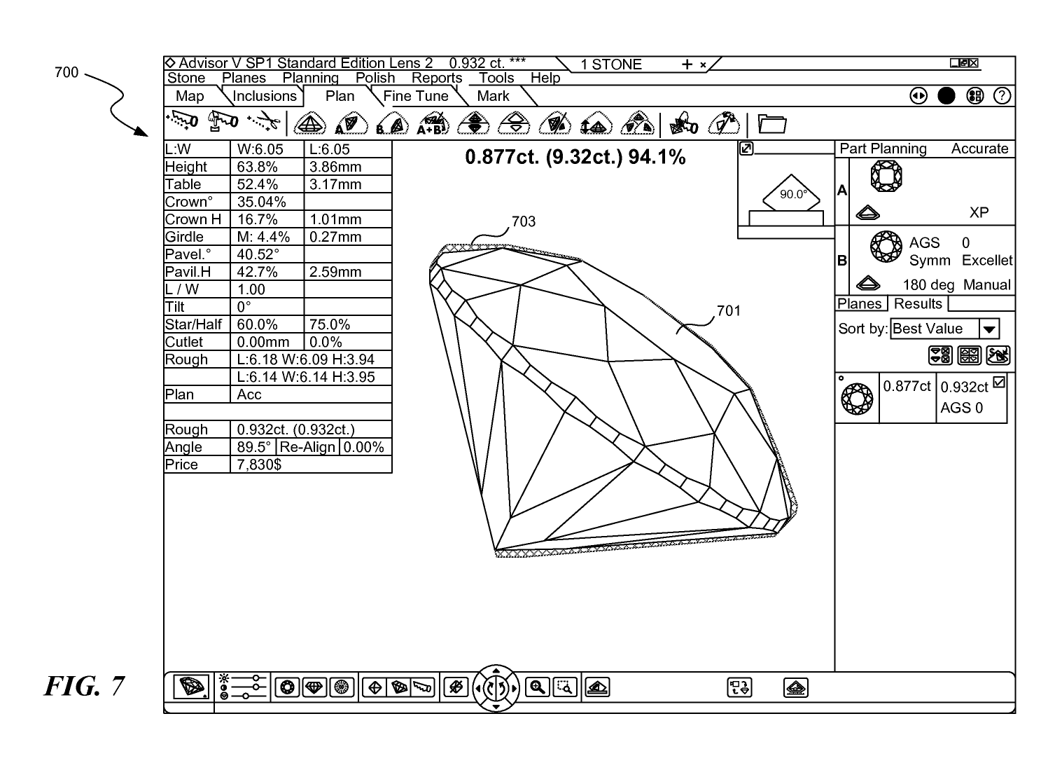

In block 412, the recut potential of the 3D model is analyzed. For example, particular instructions regarding cut and symmetry parameters can be provided. A fourth script can then utilize the virtual model 601 to project the weight of the recut polished diamond given those particular constraints. For example, if the candidate diamond has a “Fair Cut Grade,” the fourth script may project the resulting recut polished diamond to have a “Very Good Cut Grade” or an “Excellent Cut Grade,” depending on the input instructions. In general, the better the grades, the more weight the diamond will lose in the recut process. The fourth script may also consider experimental history to determine a margin of error to associate with the calculated recut. FIG. 7, for example, illustrates a graphical user interface 700 of a recut analysis software that can be utilized by the fourth script. The projected recut 701 is illustrated, as well as a shaded region 703 reflecting the portion of the candidate diamond that will be removed in the proposed recut. Although this embodiment illustrates recutting a polished diamond, in other embodiments one or more diamonds can be cut from a single rough diamond. In some embodiments, the proposed recut can be calculated automatically based upon a variety of selected parameters, such as symmetry, desired cut, etc.

Routine 400 continues in block 414 by outputting the recut potential over a network. For example, the recut potential can be provided electronically to a purchaser or other entity. Based on the proposed recut, the fair market value of the candidate diamond can be determined. In some embodiments, a fifth script can determine a fair market value for the recut diamond by searching for similar diamonds for sale and comparing prices for the similar diamonds. In some embodiments, the fifth script can automatically estimate a fair market value based on the identified parameters of the recut diamond, such as the symmetry, grade, size, etc. The determined fair market value can then be compared with the price of the identified candidate diamond. Based on the comparison, the user may determine whether to purchase the candidate diamond for actual recutting. In block 416, routine 400 ends.

Although certain embodiments disclosed herein relate to recutting polished diamonds, in some embodiments a rough diamond can be analyzed similarly for potential as a cut and polished diamond. In some embodiments both the size and shape of the candidate diamond and the size and shape of the proposed recut can vary widely. As noted previously, there are numerous circumstances under which recutting can be profitable (e.g., improving the cut grade of the diamond to correct original manufacturer errors or defects, if a manufacturer has a distribution channel that has selling prices that are higher than the general market, or if a manufacturer has a distribution channel for a proprietary shape that can be cut from traditional shapes). These various options can be incorporated into the fair market analysis in block 414.

In some embodiments, the routine 400 can include identification and analysis of multiple candidate diamonds at once and in parallel. For example, in some embodiments the routine 400 may identify and evaluate tens, hundreds, or thousands of diamonds to identify recut candidates. In some embodiments, one or more of the first, second, third, and fourth scripts, and the shape builder, may operate on a single computing device or on various different computing devices which are in network communication with one another. In some embodiments, the technical process routine 400 may be integrated into a workflow automation system and may not simply trigger a cutting process but may also automatically trigger a purchasing process based on a comparison of the determined fair market value with the price of the candidate diamond. In some embodiments, the routine 400 may further automatically execute the purchase without the need for human intervention. In these embodiments, the routine 400 not only automatically identifies and evaluates candidate diamonds, but also selects and purchases a subset of those which are projected to be profitably recut, as well as providing an output of the proposed recut for each purchased diamond. A recut of the purchased diamonds may then be executed (for example using a computer based cutting machine) based on the proposed recut. This end-to-end automation may increase the efficiency and profitability of the process beyond reducing energy and transport costs in the analyses phase or wastage in the cutting phase.

Several embodiments of the disclosed technology are described above in reference to the figures. The computing devices on which the described technology may be implemented may include one or more central processing units, memory, input devices (e.g., keyboard and pointing devices), output devices (e.g., display devices), storage devices (e.g., disk drives), and network devices (e.g., network interfaces). The memory and storage devices are computer-readable storage media that can store instructions that implement at least portions of the described technology. In addition, the data structures and message structures can be stored or transmitted via a data transmission medium, such as a signal on a communications link. Various communications links may be used, such as the Internet, a local area network, a wide area network, or a point-to-point dial-up connection. Thus, computer-readable media can comprise computer-readable storage media (e.g., “non-transitory” media) and computer-readable transmission media.

Although the subject matter has been described in language specific to structural features and/or methodological acts, it is to be understood that the subject matter defined in the appended claims is not necessarily limited to the specific features or acts described above. Specific embodiments and implementations have been described herein for purposes of illustration, but various modifications can be made without deviating from the scope of the embodiments and implementations. The specific features and acts described above are disclosed as example forms of implementing the claims that follow. Accordingly, the embodiments and implementations are not limited to those explicitly described herein.

I/We claim:

1. A system for improving efficiency in diamond analysis systems by transforming diamond measurement information into a model for analyzing a candidate diamond, the system comprising:
an interface configured to receive, from a diamond supplier, through a network, measurement information for a plurality of diamonds;
a candidate selection module configured to automatically identify at least one candidate diamond from among the plurality of diamonds;
a modeling module configured to automatically generate a virtual model of the at least one candidate diamond based at least in part on the received measurement information; and
a recut analysis module configured to automatically analyze the virtual model to determine a recut potential,
wherein the interface is further configured to provide the recut potential to a manufacturer, through the network.

2. The system of claim 1, further comprising a recut module configured to cut the candidate diamond based on the determined recut potential.

3. The system of claim 1 wherein the recut analysis module is further configured to automatically determine a fair market value for a recut of the candidate diamond based on the determined recut potential.

4. The system of claim 3 wherein the recut analysis module is further configured to automatically advise a purchase of the candidate diamond based on the determined fair market value.

5. The system of claim 1 wherein the measurement information comprises a qualitative assessment of the at least one candidate diamond.

6. A method, performed by a computing device having a processor, for analyzing a diamond, the method comprising:
receiving diamond inventory information for a plurality of diamonds;
automatically identifying at least one candidate diamond from among the plurality of diamonds;
obtaining measurement information associated with the at least one candidate diamond; and
based at least in part on the obtained measurement information, automatically generating a virtual model of the at least one candidate diamond.

7. The method of claim 6, further comprising automatically analyzing the virtual model of the at least one candidate diamond to determine a recut potential.

8. The method of claim 7, further comprising cutting the at least one candidate diamond based on the determined recut potential.

9. The method of claim 8, wherein the at least one candidate diamond has a first grade and wherein the recut candidate diamond has a second grade, the second grade higher than the first grade.

10. The method of claim 7, further comprising automatically determining a fair market value for a recut of the candidate diamond based on the determined recut potential.

11. The method of claim 10, further comprising automatically advising a purchase of the candidate diamond based on the determined fair market value.

12. The method of claim 6 wherein obtaining measurement information associated with the at least one candidate diamond comprises extracting measurement information from the diamond inventory information.

13. The method of claim 12 wherein obtaining measurement information associated with the at least one candidate diamond further comprises calculating additional measurement information based on the extracted measurement information.

14. The method of claim 6 wherein the measurement information comprises a qualitative assessment of the at least one candidate diamond.

15. A computer-readable medium storing computer-executable instructions for evaluating a diamond, the computer-executable instructions comprising instructions that, when executed by a computing system having a processor, cause the computing system to perform operations, the operations comprising:
receiving diamond inventory information for a plurality of diamonds;
automatically identifying at least one candidate diamond from among the plurality of diamonds;
obtaining measurements associated with the at least one candidate diamond; and
based at least in part on the obtained measurements, automatically generating a virtual model of the at least one candidate diamond.

16. The computer-readable medium of claim 15 wherein the operations further comprise automatically analyzing the virtual model of the at least one candidate diamond to determine a recut potential.

17. The computer-readable medium of claim 15 wherein the operations further comprise automatically determining a fair market value for a recut of the candidate diamond based on the determined recut potential.

18. The computer-readable medium of claim 17 wherein the operations further comprise automatically advising a purchase of the candidate diamond based on the determined fair market value.

19. The computer-readable medium of claim 15 wherein the obtaining measurements associated with the at least one candidate diamond comprises extracting measurement information from the diamond inventory information.

20. The computer-readable medium of claim 19 wherein obtaining measurements associated with the at least one candidate diamond further comprises calculating additional measurement information based on the extracted measurement information.
